(19) United States
(12) Patent Application Publication
(10) Pub. No.: US 2007/0185395 A1
(43) Pub. Date: Aug. 9, 2007

(54) METHOD FOR IMPLEMENTATION OF A MEDICAL EXAMINATION ON A PATIENT USING A CONFIGURABLE MEDICAL EXAMINATION APPARATUS

(76) Inventors: Karlheinz Glaser-Seidnitzer, Furth (DE); Mike Muller, Mohrendorf (DE); Elmar Seeberger, Weissenburg (DE)

Correspondence Address: SCHIFF HARDIN, LLP;PATENT DEPARTMENT, 6600 SEARS TOWER, CHICAGO, IL, 60606-6473, US

(21) Appl. No.: 11/620,152

(22) Filed: Jan. 5, 2007

(30) Foreign Application Priority Data
Jan. 5, 2006 (DE) 10 2006 000 928.2

(51) Int. Cl. A61B 5/05

(52) U.S. Cl. 600/410

(57) ABSTRACT

In a method for implementation of a medical examination on a patient using a configurable medical examination apparatus, a reconstruction document is loaded into the program controller of the examination apparatus, and the reconstruction document is stored in a basic measurement forming the basis of an intended repeat measurement, and the repeat measurement is implemented by automatic execution of the measurement protocols stored in the reconstruction document with the measurement parameter configuration likewise stored in the reconstruction document and forming the basis of the basic measurement.

### BACKGROUND OF THE INVENTION

1. Field of the Invention

The present invention generally concerns medical imaging (for example a magnetic resonance tomography (MRT) apparatus for examination of patients. The present invention in particular concerns a method as well as a device for conducting a medical examination of a patient by means of a configurable medical examination apparatus.

2. Description of the Prior Art

Magnetic resonance tomography is a method for medical diagnostics that is primarily characterized by a high contrast resolution capability for soft tissue. Since it allows the structures of the human body to be depicted to the greatest possible extent and in detail, it has become widely accepted and has been shown to be superior in many cases relative to other imaging modalities. The acquisition of structures and organs of the human body predominantly ensues by the acquisition of slices of the human body. In order to completely acquire the region of the human body that is desired for a medical examination, the acquisition of a number of such slices is required. Furthermore, it is necessary to adapt the acquisition of the individual slices to the particular conditions that are required for a specific medical examination. It thus can be required to make a specific structure of the human body presentable in an intensified manner using a contrast agent. As a further example it is possible to show moving fluids and fluids in general (such as, for example, blood in blood vessels) in a particularly high-contrast manner. For this purpose, the corresponding parameters and specifications are set in a control program before the acquisition of a specific slice exposure, the control program operating the magnetic resonance tomography device. Due to the large number of medical examination possibilities that are enabled by magnetic resonance tomography, there are is a very large number of different possibilities for acquiring magnetic resonance data that are respectively optimized for the desired examination. Operating personnel accordingly must enter a number of parameters and settings into the control program at the magnetic resonance tomography device for a specific, desired medical examination as well as for the region of the human body to be examined.

According to the prior art, there is a software architecture for MRT apparatuses that, in combination with the corresponding hardware components, makes the examination-specific configuration, the image processing and the image data administration of an MRT apparatus easier for the user. Such a software architecture is implemented, for example, in MRT apparatuses from the company Siemens under the name “Phönix” and is roughly described below using the format in FIG. 2 (a more detailed representation ensues using FIGS. 3 and 4).

The MRT apparatus 14 is equipped with an examination databank 15 that contains a number of possible measurement protocols. The user can have these displayed in a window (pop-up window) at the user screen 24 in a step S1 via the examination browser 16, make a selection via the user screen 24, and load these selected measurement protocols into the program controller 17 according to step S2. The window of the program controller 17 is likewise simultaneously displayed at the user screen 24. Furthermore, the user now has the possibility to make changes to the measurement parameter configuration of a selected measurement protocol (not shown). If the user initiates the measurement according to step S3, a reworking of the selected, possibly manually adapted measurement protocols ensues in the measurement system 18, and the images (possibly an image series) generated by the image computer 18 according to step S4 are stored in an image data bank 19. The image data bank 19 effectively corresponds to a fixed disk storage of a PC or of a workstation. The user can now have the acquired image data displayed (likewise in a window on the user screen 24) via a patient browser 20 according to step S5. With the present MRT software (Siemens corporation: Phönix) it is now possible to have the measurement that led to a specific image or, respectively, to a specific image series repeated exactly by, according to step S6, the user dragging the aforementioned image (the image series) or an icon associated with this image (or image series) onto an interactive button in the window of the program controller 17 by a drag-&-drop operation. This step S6 (the mouse-related transfer of a measurement parameter configuration to a measurement protocol by drag-&-drop) is described in detail in DE10118194A1 (“Rekonstruktion von Messprotokollen und Messparamatern aus gemessenen Bildern bei MR-Anlagen” (“Reconstruction of measurement protocols and measurement parameters from measured images in MR systems”) R. Schneider and M. Müller).

It is frequently the case, however, that not just one single measurement based on a single measurement protocol is conducted on a patient, but rather (in the framework of a disease-specific study) a procedure known as a comprehensive measurement program is conducted, composed of multiple (normally different) measurement protocols to be executed in succession, including patient relocation by table displacement and possibly different measurement pauses. On the basis of the result of such a measurement program the user (i.e. the radiologist or the treating physician) forms a diagnosis that in turn forms the basis for a more or less time-consuming therapy. It is thereby necessary to re-examine the patient at medium or long-term time intervals in order to be able to monitor the course of the illness or the course of the therapy on the basis of comparison images, known as a follow-up study.

The problem for the operator of the MRT apparatus is to determine the measurement program used for the first time with the respective measurement protocols manually adapted under the circumstances during the examination. Conventional examination systems do not allow the operator to exactly regain or to derive the previous, customized measurement program from the stored data of the first examination or of a preceding examination (thus images and/or image series that are stored in the image databank 19). In larger practices and clinics the problem is made even more severe because such examinations are planned on an RIS 23 (radiological information system), but the actual examination is then conducted by different personnel on the respective medical examination apparatus 14 (known as a “medical imaging modality”, here the MRT apparatus). The Phönix software, as a part of the operating system software of Siemens MRT apparatuses (syngoMR software), presently allows a corresponding executable measurement protocol to be reconstructed only with regard to a single series or an image that is part of a series. For this purpose, the user sits at the MR system 14 and the patient must already be registered, i.e. an examination of the patient by the user is already in progress.

### SUMMARY OF THE INVENTION

An object of the present invention is to provide a method as well as a magnetic resonance tomography apparatus for implementation of the method that enable, in a simple manner, the exact repetition of a comprehensive MRT examination, for example in the framework of a running therapy or a follow-up examination on a patient, even after large time spans and given arbitrarily complex measurement parameter configuration.

This object is achieved according to the invention by a method for implementation of a medical examination on a patient using a configurable medical examination apparatus, including loading a reconstruction document into the program controller of the examination apparatus, the reconstruction document being stored in a basic measurement forming the basis of an intended repeat measurement, and implementing the repeat measurement by automatic execution of the measurement protocols stored in the reconstruction document with the measurement parameter configuration likewise stored in the reconstruction document and forming the basis of the basic measurement.

According to the invention, the reconstruction document includes the measurement program used in the basic measurement as well as the measured result images and/or result series.

The user can influence the repeat measurement by eliminating irrelevant measurement protocols in the measurement program after the loading but before the repeat measurement.

The loading of the reconstruction document likewise advantageously ensues automatically on the basis of a linking of the patient data (input or retrieved in the repeat measurement) with the corresponding patient data of the patient registration that occurred in the basic measurement.

In a further embodiment of the inventive method, the linking is initiated manually by the user by a drag-&-drop operation, dragging of a graphical element symbolizing the basic measurement from the patient browser of the examination apparatus into the program controller of the of the examination apparatus.

The loading of the reconstruction document likewise advantageously ensues automatically from a local databank of the examination apparatus, from a transportable data storage (such as, for example, a CD) or if applicable from a long-term storage (such as, for example, the archive of a PACS) via an RIS.

According to the invention the examination apparatus is fashioned as a magnetic resonance tomography apparatus, ultrasound apparatus, C-arm, CT apparatus, PET apparatus, SPECT apparatus, etc.

The above object also is achieved by a device suitable for implementation of the above-described method.

The above object also is achieved by a computer-readable medium encoded with a data structure, the data structure causing an imaging modality to operate as described above, the data structure runs in a computer of the modality.

### DESCRIPTION OF THE DRAWINGS

FIG. 1 schematically illustrates a magnetic resonance tomography apparatus according to the present invention.

FIG. 2 shows, in a flowchart, the functionality of the Phönix technology internal to the MRT apparatus according to the prior art.

FIG. 3 shows, in a flowchart, the functionality of the inventive Auto-Phönix technology given connection of the MRT apparatus to a PACS.

FIG. 4 shows the functionality of the inventive Auto-Phönix technology in a flowchart, in an embodiment wherein the connection of the MRT apparatus to a PACS ensues via an RIS.

### DESCRIPTION OF THE PREFERRED EMBODIMENTS

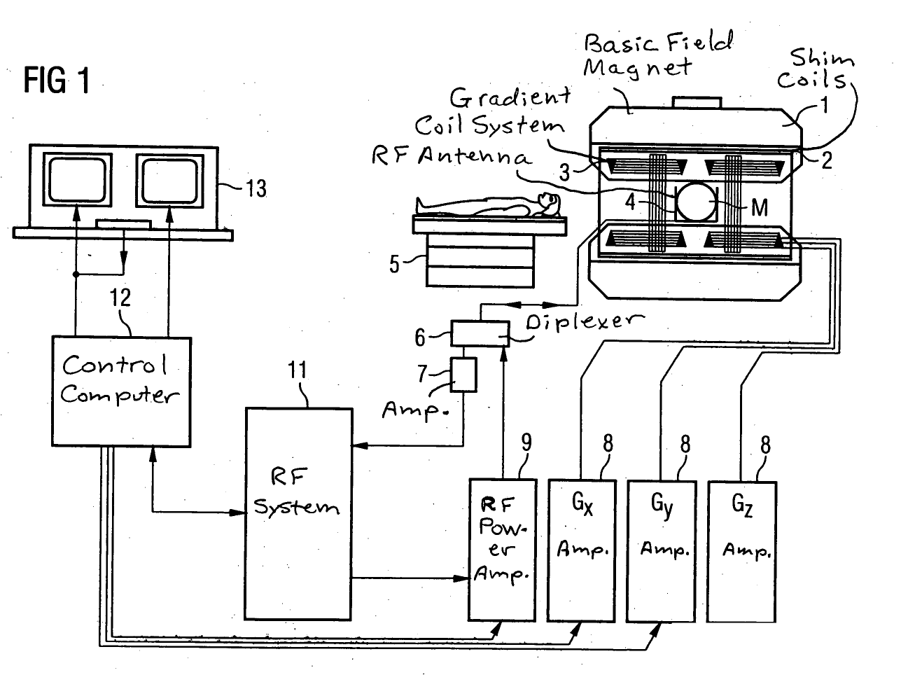

FIG. 1 is a schematic representation of a magnetic resonance tomography apparatus for generation of a magnetic resonance image of a subject, with which the inventive method is applied. The design of the magnetic resonance tomography apparatus corresponds to that of a conventional magnetic resonance tomography apparatus, with the exceptions described below. A basic field magnet 1 generates a temporally constant strong magnetic field for polarization or alignment of the spins in the examination region of a subject such as, for example, of a portion of a human body to be examined. The high homogeneity of the basic field magnet that is required for the magnetic resonance data acquisition is defined in a spherical measurement volume M into which the person to be examined is introduced on a subject table 5 that can be moved into the magnetic resonance tomography apparatus such that the parts of the human body to be examined are located in the measurement volume M. In order to satisfy the homogeneity requirements for the magnetic field and to eliminate temporally non-varying influences, shim plates made of ferromagnetic material are mounted at suitable points. Temporally-varying influences are eliminated by shim coils 2. A cylindrical gradient coil system 3 that has three windings is inserted into the basic field magnet 1. Each winding is supplied with current by an amplifier 8 to generate a linear gradient field in one direction of a Cartesian coordinate system. The first winding of the gradient coil system 3 generates a gradient Gx in the x-direction, the second winding generates a gradient Gy in the y-direction and the third winding generates a gradient Gz in the z-direction. The volume to be measured (established by the selected resolution) is spatially encoded by the gradient fields, the encoding service to establish points of the later image.

Located within the gradient field system 3 is a radio-frequency (RF) antenna 4 that converts the radio-frequency pulses (emitted by a radio-frequency power amplifier 9 via a transmission/reception diplexer 6) into an alternating magnetic field for excitation of the nuclei and “flipping” alignment of the nuclear spins of the subject to be examined or of the region of the subject to be examined. The alternating field originating from the precessing spins (i.e. normally the spin echo signals caused by a pulse sequence composed of one or more radio-frequency pulses and one or more gradient pulses) is also converted by the RF antenna 4 into a voltage that is supplied via the transmission/reception diplexer 6 as well as via an amplifier 7 to a radio-frequency system 11.

In a control computer 12 an image is generated from measurement data acquired in such a manner. The administration of the image data and of the acquisition parameters (with which the interaction of the gradient fields and the radio-frequency system 11 is controlled), necessary for the protocol of the individual measurements, also ensues in the control computer 12. The control computer 12 in particular controls the sequence order, i.e. the time-accurate switching of the gradients, the emission of the radio-frequency pulses with defined phase and amplitude as well as the acquisition of the magnetic resonance signals. The selection of corresponding sets of acquisition parameters for generation of a magnetic resonance image as well as the representation of the generated magnetic resonance image likewise ensue via the control computer 12, which has a terminal 13, a keyboard and one or more screens.

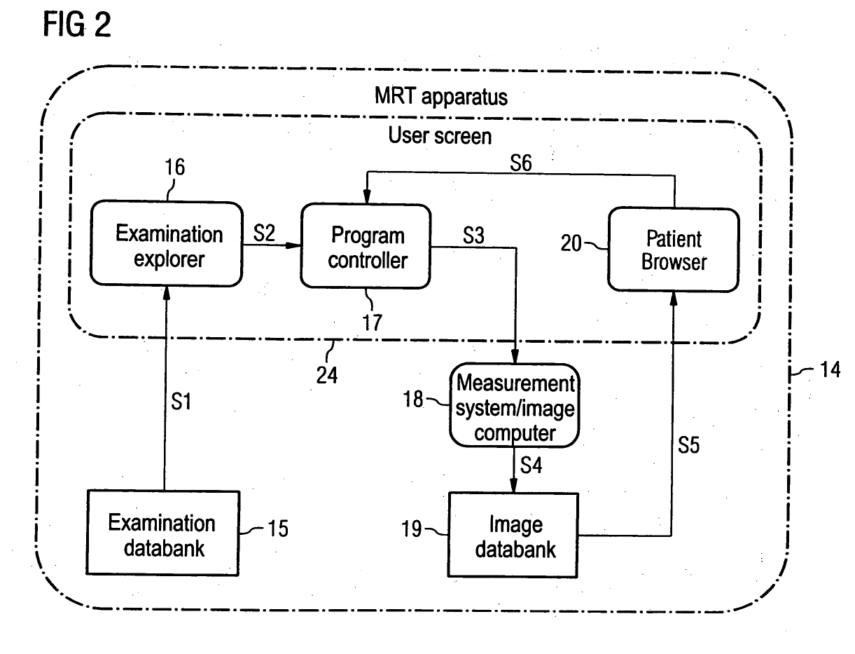

Those components of an MRT apparatus (just described) that are essentially responsible for the data flow, the data retrieval and data storage (it measurement data, measurement protocol data, or image data) are shown in FIGS. 2, 3 and 4 (FIG. 2 was already briefly described above), including the user interfaces on the user screen 24 via which the user can influence the data processing.

The starting basis is the examination databank UDB 15 that represents a data storage for a number of measurement protocols and, due to the various usage possibilities of an MRT apparatus 14, is strictly hierarchically structured in the following manner:

The UDB 15 contains “regions” corresponding to an anatomical division of the human body; a “region” encompasses “examinations” corresponding to possible diagnostic questions; an “examination” encompasses “measurement programs” corresponding to a specific procedure in an examination; and a “measurement program” encompasses “measurement protocols” corresponding to an accentuation in the sequence of a “measurement program”. A “measurement protocol” (as smallest unit) encompasses all measurement parameters necessary for the control of a measurement.

The structure of the UDB 15 can be displayed to the user in a step S1 via the examination explorer 16 on the user screen 24, and individual measurement protocols (normally, however, an available measurement program corresponding to the intended examination) can be entered into the program controller 17 according to a step S2.

Such a measurement program includes a number of measurement protocols that are possibly temporally spaced by pauses, and as such form a sequence known as the measurement queue.

The user normally also has the possibility to influence the measurement queue via the window of the program controller 17 and will also do this, for example, by modifying the measurement parameter configuration of one or more protocols and/or modifies the pauses between the individual measurements. The intervention into individual elements of the measurement queue is typically based on the level of experience of the user, and the individual, patient-specific clinical situation is taken into account in the modification of the measurement queue.

After the initiation of the measurement according to step S3 and the measurement itself (which ensues via the measurement system 18 according to step 4 by a chronological execution of the measurement queue (modified where possible)), the image data generated by the image computer 18 are stored in the image data bank 19 in the form of individual images or an image series. The image data bank 19 represents a primary data store for the image data generated in the measurement.

The image databank 19 is also hierarchically structured: it includes on the uppermost level “patients”; a “patient” encompassed by “studies”; a “study” encompasses “series”; and a “series” encompasses “images”. This structure also can be displayed to the user according to step S5 via the patient browser 20 in a pop-up window on the user screen 24 for immediate diagnosis by the corresponding images being read out from the image data bank 19.

Since the image data bank 19 generally has only a limited storage capacity, usually in smaller radiological practices image data that are not immediately relevant are burned on a CD/DVD and are stored and retained within the practice, for example in a CD/DVD archive cabinet. In larger practices and hospitals the image data bank 19 of an MRT modality 14 is connected to a long-term image archive 22 of a PACS 21 (Picture Archiving and Communicating System) which normally includes magnetic storage media (for example magnetic tapes) that, although they are stored at a protected location (for example a vault), are retrievable by networking via a LAN (local area network) from the image workstation at the MRT 14.

A larger radiological practice or a hospital (as already mentioned) is connected to a radiological information system 23 (RIS) and data access to the image data of the long term PACS archive 22 thereof or to another practice or clinic ensues via the RIS 23.

The present invention involves the design of the software architecture of an MRT system 14, possibly with connection to further information systems (PACS 21, RIS 23) such that, in the framework of a maximally complex follow-up study (repeated multi-stage examination of a patient on an MRT apparatus 14, the measurement program forming the basis of the repeat measurement is exactly retrieved or can be reconstructed, which conventionally is possible only with a markedly high expenditure, or is not possible at all in the case of the present Phönix software.

For this purpose, more information will inventively be stored in the aforementioned image storage media upon storage of the examination results of a follow-up study. in accordance with the invention, not only images, image series and the associated measurement protocols are thus stored in the image databank on a CD/DVD or in a PACS archive 22, but rather also further data required for an automated workflow. These data are, for example, all demographic patient data such as name, birth name, weight, age etc., as well as characteristic measurement data such as measurement pauses, table displacement, breathing instructions or other instructions (eyes open, eyes closed, turn head, etc.).

For these follow-up study data, during or after the measurement a new, separate document is inserted into the structure of the image databank 19, this document additionally appears on the user screen 24 with the previous typically-displayed data upon being called by the patient browser 20.

For example, according to the invention the following structure results for the image databank 19 or for the documents stored there.

The image databank 19 encompasses “patients”; a “patient” encompasses “studies”; and a “study” encompasses “report series”. According to the invention a “reconstruction document” (Phönix: evidence document) is entered into such a “report series”. This “reconstruction document” serves as a data repository for the employed measurement program, for relevant result images and/or relevant result series as well as optionally for the finding of the radiologist. “Relevant” means that only meaningful (informative) results are stored and qualitatively unusable images/image series are discarded (thus deleted) at the outset. In this reconstruction document, data from different segments of an examination are thus collected (for example the user selection of the measurement program with the respective measurement parameter configuration of the respective measurement protocols, images of the patient and the finding that typically is only generated when the patient is long gone). This makes it possible (depending on the type of the implementation of these reconstruction documents) to be able, in later follow-up examinations, to recapitulate the basic examination exactly and in a user-friendly manner (for example via a mouse click on a correspondingly-placed button of an activated window on the user screen 24).

Essentially two cases can be differentiated with regard to the follow-up examinations:

I) MRT System without RIS 23 (Connection)

If the patient comes into a practice without an RIS connection for a follow-up examination, according to step S5 the reconstruction document which was generated and stored in the first examination must be recalled.

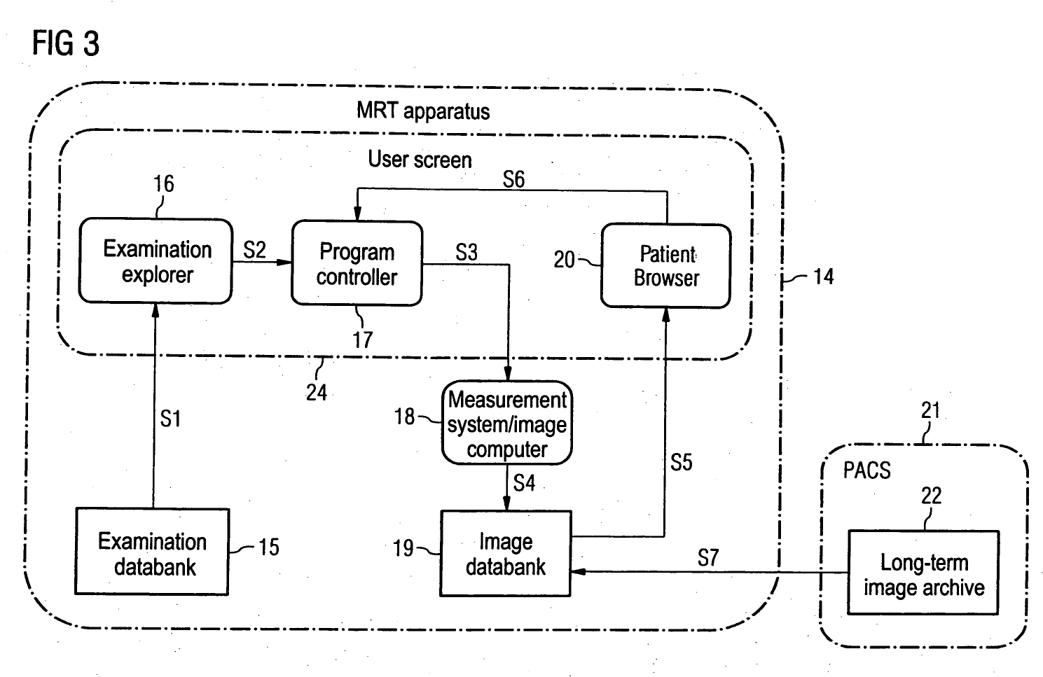

If this reconstruction document is no longer present in the local image databank 19 of the MR system 14 (this is usually the case after longer periods of time), according to step S7 (FIG. 3) it must be loaded from the long-term image archive 22 of the PACS 21 or (in the event that the connection to such an archive does not exist) directly from a CD/DVD. In a further step S6 of the follow-up examination the reconstruction document is loaded into the measurement queue of the program controller 17 by drag-&-drop (mouse-based, as presently in Phönix for images and image series (see above)), so the measurement program used the first time is exactly reconstructed with all measurement protocols, measurement parameters and further details.

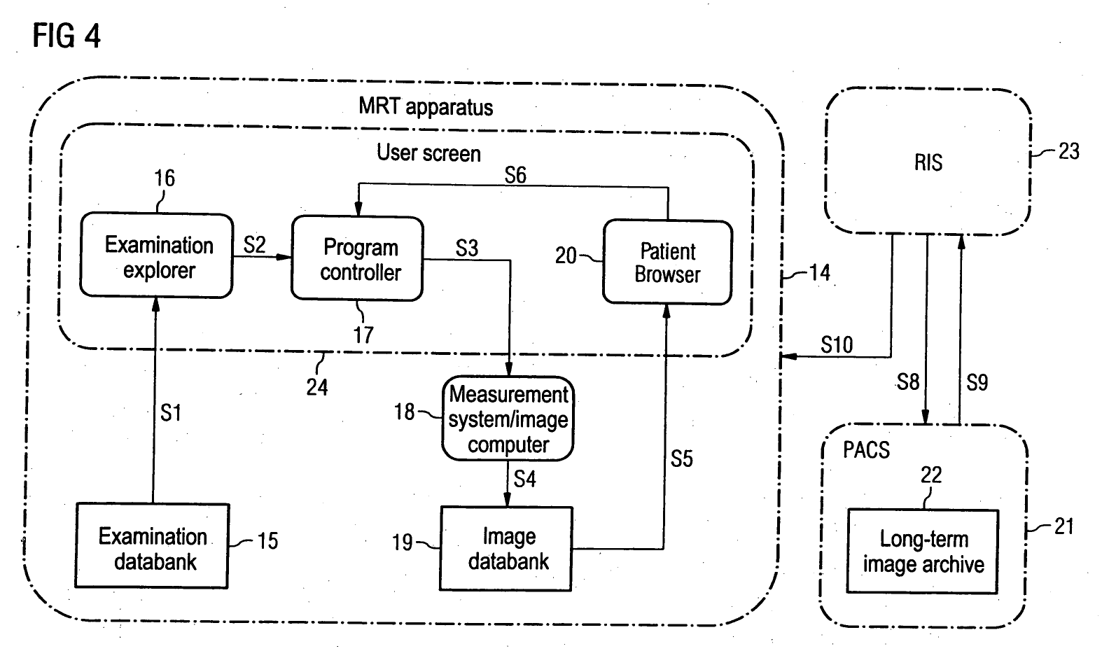

II) MRT System with RIS 23 (Connection) (FIG. 4)

More elaborate examinations (for example on MRT apparatuses at an RIS 23 (terminal) are planned (FIG. 4) in larger (radiology) practices or in well-equipped clinics. This means that it is established there which patient is examined when and at which modality, whereby the patient registration (i.e. the input of the patient demography) also ensues on site. On the basis of this information the RIS user is able to be informed (according to step S8) whether a prior examination already exists in the PACS 21 with regard to this patient. If this is the case, the patient documents or the patient structure (including the reconstruction document according step S9 and step S10) are transferred over the RIS from the PACS 21 to the MR apparatus 14 and are displayed on the user screen 24 via the patient browser 20 according to step S5.

The user at the MR apparatus 14 is then able to survey the MR protocols and to decide which measurement protocols have lead to relevant results and therefore should be reused.

The registration of the patients at the MR apparatus 14 is also confirmed, which leads to the situation that the selected measurement protocols of the underlying measurement program are thus loaded into the program controller 17 on the basis of the reconstruction document, such that a measurement queue is generated automatically that—except for the eliminated measurement protocols—exactly corresponds to the measurement queue of the first or basic measurement that occurred in the framework of the prior examination.

This has the result that the user does not manually search in the local image databank 19 for all usable images or series and, where possible (in the case without a PACS), does not have to search for CDs or DVDs and insert these in the event that the data are no longer present in the local image databank 19, in order to then individually load the MR measurement protocols into the measurement queue of the program controller 17 by means of Phönix.

Based on the reconstruction document the repeat measurement can run automated in a simple manner after a patient registration confirmation. This inventive procedure or the software underlying this procedure is designated as Auto-Phönix.

In both cases I) and II) a repeat measurement exactly corresponding to a first or basic measurement can be implemented without noteworthy personnel use, which repeat measurement leads to technically identical comparison images/comparison image series due to the same measurement queue and ensures the doctor a best-possible assessment of the temporal course of disease.

Advantages of the inventive method can be summarized as follows:
A reduction of the personnel (operating and time) expenditure results in a follow-up examination via the automatic loading of the measurement program of the prior examination forming the basis of the follow-up examination.
The selection of relevant measurement protocols avoids unnecessary examination steps in follow-up examinations and therewith [sic] a connected cost-savings since only necessary examination steps may be billed.
Follow-up examinations can also be implemented on a simple basis by means of Auto-Phönix when the measurement protocols with the configuration of the basic measurement are no longer present at all in the examination databank since all information are stored in the reconstruction document.

The invention has been explained in the context of magnetic resonance tomography, but it is apparent that the present invention is also applicable for other medical examination methods (for example CT, PET, SPECT, US, AX, etc.) in which apparatus parameters for examinations must be set in order to be able to achieve examination results that can be correlated.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for implementing a medical examination on a patient using a configurable medical examination apparatus, comprising the steps of:
in a basic examination of a patient using a configurable medical examination apparatus generating a reconstruction document having stored therein all measurement protocols and measurement parameter configurations, including manually-entered modifications to said protocols and parameters, used in the basic examination;
for a repeat examination of the patient using a configurable medical examination apparatus corresponding to the configurable medical examination apparatus used for the basic examination, having a program controller, loading said reconstruction document into the program controller; and
executing said repeat measurement with said configurable medical examination apparatus corresponding to the medically configurable examination apparatus used for the basic examination, by automatic execution of said measurement protocols and parameter configurations and modifications stored in said reconstruction document.

2. A method as claimed in claim 1 comprising additionally storing image information in said reconstruction document selected from the group consisting of an image obtained as a result of said basic examination and an image series obtained as a result of said basic examination.

3. A method as claimed in claim 1 comprising allowing manual modification of said repeat measurement by a user after loading said reconstruction document into said program controller, but before implementing said repeat measurement.

4. A method as claimed in claim 1 wherein the step of loading said reconstruction document comprises linking patient data with said reconstruction document, and entering patient data obtained from said patient prior to implementing said repeat measurement into said program controller, and retrieving said reconstruction document based on said patient data and automatically loading the retrieved reconstruction document into said program controller.

5. A method as claimed in claim 4 comprising linking said patient data via a user interface by a drag-and-drop operation of an input element, by dragging a graphical element at a display associated with said program controller, symbolizing said basic measurement.

6. A method as claimed in claim 1 comprising storing said reconstruction document in a storage medium, selected from the group consisting of portable data storage media and long-term storage media, and wherein the step of loading said reconstruction document comprises loading said reconstruction document into said program controller from said storage medium.

7. A method as claimed in claim 1 comprising conducting said basic examination and said repeat examination using, as said configurable medical examination apparatus, an apparatus selected from the group consisting of magnetic resonance tomography apparatuses, ultrasound apparatuses, C-arm x-ray apparatuses, computed tomography apparatuses, PET apparatuses, and SPECT apparatuses.

8. A configurable medical examination apparatus comprising:
a data acquisition unit configured to interact with a patient to acquire medically-relevant data from the patient; and
a program controller connected to said data acquisition unit, said program controller being loaded with and programmed by a reconstruction document generated in a previously-conducted basic examination of the patient, said reconstruction document containing all measurement protocols and parameter configurations, and any manually-made modifications to said measurement protocols and parameter configurations, used in said previously-conducted basic examination, and said program controller implementing said repeat measurement with said data acquisition unit by automatically executing the measurement protocols and parameter configurations and modifications stored in said reconstruction document.

9. A computer readable medium encoded with a data structure, said computer readable medium being loadable into a program controller of a medically configurable examination apparatus, and said data structure causing said program controller to automatically execute a repeat measurement to acquire medically-relevant data from a patient, using medically configurable examination apparatus, according to a reconstruction document contained in the data structure that comprises measurement protocols and measurement parameter configurations and manually-made modifications to said measurement protocols and parameter configurations, from a previously-conducted basic examination of the patient using a configurable medical examination apparatus corresponding to said medically configurable examination apparatus operated by said program controller.
